(19) United States
(12) Patent Application Publication
(10) Pub. No.: US 2023/0120351 A1
(43) Pub. Date: Apr. 20, 2023

(54) Filter Table

(71) Applicant: Peter J. Luciani, JR., Buzzards Bay, MA (US)

(72) Inventor: Peter J. Luciani, JR., Buzzards Bay, MA (US)

(21) Appl. No.: 17/448,130

(22) Filed: Sep. 20, 2021

Related U.S. Application Data
(60) Provisional application No. 63/080,184, filed on Sep. 18, 2020.

(51) Int. Cl. A61L 9/014; A61L 9/14; A61L 9/18; A61L 9/22

(52) U.S. Cl. CPC A61L 9/014; A61L 9/14; A61L 9/18; A61L 9/22; A61L 2209/14; A61L 2209/22; A61L 2209/111; A61L 2101/34

(57) ABSTRACT

A decontaminating filter table for decontaminating exhaled or ambient air is provided. The table includes an input air grille; a first air trough in fluidic connection with the input air grille; a second air trough in fluidic connection with the first air trough; and a HEPA filter for filtering the decontaminated air. The table also includes an air mover for drawing the exhaled air through the first and second air troughs and through the HEPA filter. In addition, the table includes a scrubber for decontaminating the exhaled air and a visual/audio indicator for monitoring air flow through the first and second troughs.

### CROSS-REFERENCE TO RELATED APPLICATIONS

The present application is related to, claims the earliest available effective filing date(s) from (e.g., claims earliest available priority dates for other than provisional patent applications; claims benefits under 35 USC § 119(e) for provisional patent applications), and incorporates by reference in its entirety all subject matter of the following listed application(s) (the “Related Applications”) to the extent such subject matter is not inconsistent herewith; the present application also claims the earliest available effective filing date(s) from, and also incorporates by reference in its entirety all subject matter of any and all parent, grandparent, great-grandparent, etc. applications of the Related Application(s) to the extent such subject matter is not inconsistent herewith:
U.S. provisional patent application 63/080,184, entitled “Filter Table”, naming Peter J. Luciani Jr. as inventor, filed 18 Sep. 2020.

### BACKGROUND

### 1. Field of Use

The present invention is directed towards preventing the transmission of airborne pathogens between individuals. More specifically, the present invention is directed to apparatuses and methods designed to divert and/or to render harmless pathogens transferred by exhalation/inhalation from one person to another while at normal conversational distances apart.

### 2. Description of Prior Art (Background)

With the urgent need to prepare for potential pandemics stemming from avian influenza, swine influenza, Severe Acute Respiratory Syndrome (SARS), and other expiratory pathogenic particles, much has been done to clarify transmission mechanisms, the nature of the biological threat, and subsequent vaccine development and distribution. However, much still has to be accomplished in regards to protecting the public at large in the event of a fast moving pandemic such as COVID 19. Many of the particles concerned are less than about 1 micron in size and this is well beyond the capability of a dust mask or even a surgical mask to filter out.

Particles of less than about 5 microns in aerodynamic diameter are often termed aerosols and can be very infectious since they can travel into the alveolar region of the lungs. It is not only sneezing and coughing that discharge such pathogens into the air, but also normal exhalation releases pathogens and these tend to be in the very harmful 1 micron or less size. With normal breathing these particles are traveling at nowhere near the velocity of a cough or sneeze, but because of their small size and low settling velocity they remain airborne for considerable time and can easily reach the breathing zone of other parties within conversational distances.

Coughing or sneezing typically produce larger particles that penetrate as far as the tracheobronchial lung region and, as such, can also prove to be very infectious. These larger particles tend to drop out of the air quite rapidly but not rapidly enough so as not to be a threat at normal conversational distances (e.g. up to 3 feet, etc.). For instance, in the case of SARS, the CDC (Centers for Disease Control and Prevention) recommends keeping more than three feet apart to minimize direct droplet transfer. This is also the case for droplet transfer involving other communicable diseases. Conversational distances are typically three feet or less, so protection is also needed from droplet transfer.

While infections can occur from pathogens distributed within the general room air, the probability of such infection is lower as compared to the much higher probability of infection from pathogens directly transferred at conversational distances. Ref: Aerosol Transmission of Influenza A Virus: A review of New Studies: Raymond Tellier: J. R. Soc. 2009 6, S783-790 September 2009. A non-intrusive air barrier will eliminate this close proximity route for the spreading of infections from one individual to another.

### BRIEF SUMMARY

In one embodiment, the present invention is directed towards flexible and non-intrusive apparatuses, systems and methods to prevent direct pathogen airborne contamination from one person to another, preferably while positioned opposite, or substantially opposite from each other, such as, for example, at a desk, table or similar workstation. For purpose of this disclosure, it is understood that the term “pathogen” includes any airborne particulate including, for example, bacteria, viruses, spores, as well as any chemical molecules, such as scents and odors emitted from a person such as, for example perfume, etc.

Therefore, one embodiment of the present disclosure is directed towards a method of producing a predetermined negative stream of flowing air and directing the negative air stream to a predetermined area between individuals located a predetermined distance apart to establish an air barrier.

A further embodiment of the present disclosure is directed to an apparatus for producing a stream of flowing air between individuals, said apparatus comprising a means for producing a substantially sustained predetermined airflow to establish negative air barrier between at least two individuals spaced apart from one another by a predetermined distance. According to one embodiment, the apparatus comprising a means for depressurizing air and producing the air barrier does not impinge on the individuals, or preferably is not perceived or sensed by the individuals.

The apparatuses, systems and methods for one embodiment of the present disclosure contemplate providing negative air stream positioned between individuals on opposite sides of a table. The purpose of the negative air stream is to divert the exhaled air from each party into a filtration system and scrubber to eliminate the pathogens rather than simply diverting the pathogens into the general air space.

In another embodiment, the diverted exhaled air is diverted into a scrubber before the air stream exits the apparatus via a HEPA filter. The term scrubber refers to the introduction of any agent that renders human pathogens harmless or, at a minimum, reduces the virulence of the pathogen. For example, the scrubber includes air disinfectants that have been shown to be effective against many if not most pathogenic viruses and bacteria.

Studies in the 1940s and early 1950s “Ref: The Bactericidal Action of Propylene Glycol Vapor on Microorganisms Suspended in the Air O. H. Robertson et. al. J. Exp. MED. 1942 Jun. 1; 75(6): 593-610” showed that various glycols, were very effective in reducing airborne pathogen counts. Most particularly, propylene glycol and triethylene glycol were shown to be lethal to airborne microorganisms in low concentrations while known to have no toxicity to humans. This lack of toxicity allows propylene glycol to be used today as an additive in everything from foods, medications, toothpaste to theatrical fogs. According to the present disclosure, well under 1 gm of propylene glycol is added to the device air stream per day. This is sufficient to provide the required kill rate of pathogens in the barrier zone, but represents a miniscule concentration when diluted in the total room air. The use of a heated wick, saturated with propylene glycol, is one non-exclusive method of delivering these small amounts of disinfectant in a controllable way, since the amount of heat to accomplish this is easily provided and may be adjusted as needed. Preferably, an amount of propylene glycol is supplied to a wick from a small reservoir that can be periodically refilled or replaced as a design feature of the device. Other disinfectants, or mixtures of one or more suitable disinfectants, that are low in toxicity may also be advantageously used. Negative ion generators may also find utility for the destruction of pathogens in the barrier zone in some applications, as may the introduction of ozone and/or radiation, such as, for example UV radiation, etc.

Heating is not the only method of adding disinfectants to the air stream. Embodiments of the present disclosure further contemplate, for example, evaporation by providing extended surface area of disinfectant across which the air flows, ultrasonic addition, injection from a high-pressure disinfectant containing cartridge, micro-pump addition, etc.

### BRIEF DESCRIPTION OF THE DRAWINGS

The subject matter which is regarded as the invention is particularly pointed out and distinctly claimed in the claims at the conclusion of the specification. The foregoing and other objects, features, and advantages of the invention are apparent from the following detailed description taken in conjunction with the accompanying drawings in which:

FIG. 1 is a top view of the present invention;

FIG. 2 is a partial pictorial view of the sectional view of the invention shown in FIG. 1 along the cutting plane line B;

FIG. 3 is a partial front view of the sectional view of the invention shown in FIG. 1 along the cutting plane line B;

FIG. 4 is a front view of the present invention shown in FIG. 1;

FIG. 5 is a side view of the sectional view of the invention shown in FIG. 4 along the cutting plane line A;

FIG. 6 is a top pictorial view of the invention shown in FIG. 1; and

FIG. 7 is a partial sectional view of the invention shown in FIG. 1.

### DETAILED DESCRIPTION

The following brief definition of terms shall apply throughout the application:

The term “comprising” means including but not limited to, and should be interpreted in the manner it is typically used in the patent context;

The phrases “in one embodiment,” “according to one embodiment,” and the like generally mean that the particular feature, structure, or characteristic following the phrase may be included in at least one embodiment of the present invention, and may be included in more than one embodiment of the present invention (importantly, such phrases do not necessarily refer to the same embodiment);

If the specification describes something as “exemplary” or an “example,” it should be understood that refers to a non-exclusive example; and

If the specification states a component or feature “may,” “can,” “could,” “should,” “preferably,” “possibly,” “typically,” “optionally,” “for example,” or “might” (or other such language) be included or have a characteristic, that particular component or feature is not required to be included or to have the characteristic.

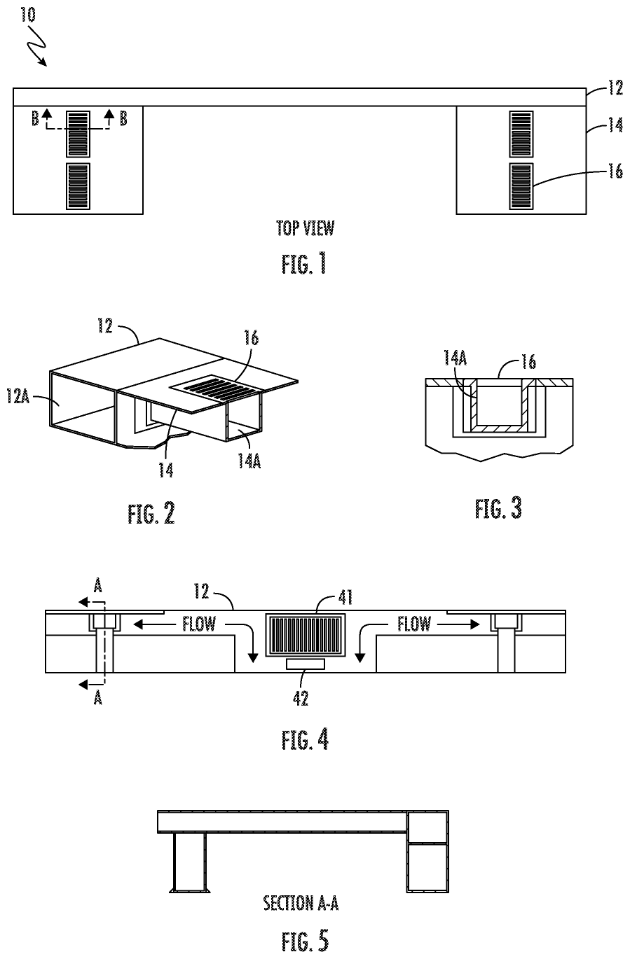

Referring now to FIG. 1 there is shown a top view of the present invention 10. Shown in FIG. 1 is base unit 12, table top grill 16 where exhaled air is drawn into a trough (shown in FIG. 2), and table top 14. It will be understood that table top grill 16 may be any suitable grill and may include preliminary air and/or sediment filtering.

Referring also to FIG. 2 there is shown a partial pictorial view of the sectional view of the invention shown in FIG. 1 along the cutting plane line B. Shown in FIG. 2 is table top trough 14A and base unit trough 12A. Exhaled air is drawn into base unit trough 12A via table top trough 14A. FIG. 3 is a partial front view of the sectional view of the invention shown in FIG. 1 along the cutting plane line B. The table top trough is typically offset from the base unit trough to help prevent sediment from entering the base trough unit. It will be understood that base unit trough 12A will normally have a removeable cover (not shown) for maintenance access to the base unit trough 12A.

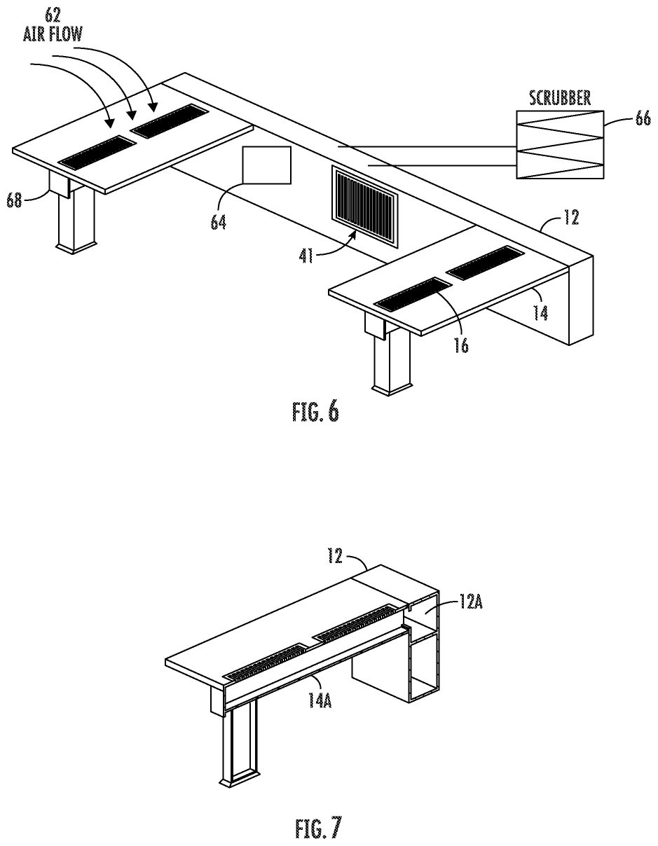

Referring also to FIG. 4 there is shown a front view of the present invention shown in FIG. 1. Shown in FIG. 4 is exhaust filter 41 and air mover 42. Air mover 42 may be any suitable air mover for creating a suitable air suction, or negative air pressure at, and above the table top grill 16. Exhaust filter 41 may be any suitable filter, such as a HEPA filter and/or a carbon filter for filtering the captured exhaust before the captured air is released back into the environment.

Referring also to FIG. 5 there is shown a side view of the sectional view of the invention shown in FIG. 4 along the cutting plane line A. Referring also to FIG. 6 there is shown a top pictorial view of the invention shown in FIG. 1. Shown in FIG. 6 is air flow 62 captured by negative air pressure or suction through table top grill 16 created by air mover 42 shown in FIG. 4. Air flow, containing contaminants, is routed through base unit trough 12A via table top trough 14A to exhaust filter 41. Before exhausting air flow 62 though exhaust filter 41 the contaminated air may be routed through scrubber 61. Removeable end plate 68 may be removed to add table extensions and/or access table top trough 14A for maintenance.

Still referring to FIG. 6 scrubber may include decontamination and/or sterilizing agents and or mechanisms such as UV or infrared light, and/or aerosolized propylene glycol for mixing with and decontaminating the contaminated air flow 62. It will be appreciated that as little as 0.5 mg. of propylene glycol per liter can almost completely sterilize an atmosphere containing hundreds of thousands of bacteria (such as Staphylococcus albus) per cubic foot. Moreover, this action occurs within a space of 15 seconds or less. Triethylene glycol, which is even less active than propylene glycol in vitro, is almost one hundred times more potent in the air. Scrubber 61 may also use microwave generator as an effective microbicide. Scrubber 61 may also include an activated carbon filter.

Also shown in FIG. 6 is visual/audio indicator 64. The visual/audio indicator 64 monitors air flow and air mover 42 operation. If the air flow through the troughs falls below an acceptable flow rate the visual/audio indicator 64 indicates the fault through a visual or audio signal. The visual/audio indicator 64 may also be Bluetooth enabled for inclusion in an internet wide maintenance program. Visual/audio indicator 64 may also include a micro total analysis system (often referred to as lab-on-a-chip) for detecting, recording, and reporting detected pathogens.

FIG. 7 is a partial sectional view of the invention shown in FIG. 1. showing the table trough 14A and base unit trough 12A.

It should be understood that the foregoing description is only illustrative of the invention. Thus, various alternatives and modifications can be devised by those skilled in the art without departing from the invention. Accordingly, the present invention is intended to embrace all such alternatives, modifications and variances that fall within the scope of the appended claims.

What is claimed is:

1. A decontaminating filter table for decontaminating exhaled or ambient air, comprising:
an input air grille;
a first air trough in fluidic connection with the input air grille;
a second air trough in fluidic connection with the first air trough;
a filter for filtering the decontaminated air; and
an air mover for drawing the exhaled air through the first and second air troughs and through the filter.

2. The decontaminating filter table as in claim 1 wherein the filter comprises a HEPA filter.

3. The decontaminating filter table as in claim 1 wherein the filter comprises an activated carbon filter.

4. The decontaminating filter table as in claim 1 further comprising a scrubber for decontaminating the exhaled air.

5. The decontaminating filter table as in claim 4 wherein the scrubber comprises sterilizing agents.

6. The decontaminating filter table as in claim 4 wherein the scrubber comprises sterilizing infrared light.

7. The decontaminating filter table as in claim 4 wherein the scrubber comprises a microwave generator.

8. The decontaminating filter table as in claim 4 wherein the scrubber comprises an activated carbon filter.

9. The decontaminating filter table as in claim 1 further comprising a visual/audio indicator for monitoring air flow through the first and second troughs.

10. The decontaminating filter table as in claim 9 wherein the visual/audio indicator comprises a micro total analysis system for detecting, recording, and reporting detected pathogens.

11. A scrubber filter table for decontaminating exhaled or ambient air, comprising:
an input air grille;
a first air trough in fluidic connection with the input air grille;
a second air trough in fluidic connection with the first air trough;
a filter for filtering the decontaminated air, wherein the filter comprises:
a HEPA filter;
an activated carbon filter;
an air mover for drawing the exhaled air through the first and second air troughs and through the filter;
a scrubber for decontaminating the exhaled air, wherein the scrubber comprises:
an activated filter;
sterilizing agents; and
sterilizing infrared light.

12. The scrubber filter table as in claim 11 wherein the scrubber comprises a microwave generator.

13. The scrubber filter table as in claim 11 further comprising a visual/audio indicator for monitoring air flow through the first and second troughs.

14. The scrubber filter table as in claim 11 wherein the visual/audio indicator comprises a micro total analysis system for detecting, recording, and reporting detected pathogens.

15. A decontaminating scrubber filter table for decontaminating exhaled or ambient air, comprising:
an input air grille;
a first air trough in fluidic connection with the input air grille;
a second air trough in fluidic connection with the first air trough;
a filter for filtering the decontaminated air, wherein the filter comprises:
a HEPA filter;
an air mover for drawing the exhaled air through the first and second air troughs and through the filter;
a scrubber for decontaminating the exhaled air, wherein the scrubber comprises:
an activated carbon filter;
sterilizing agents;
sterilizing infrared light; and
a visual/audio indicator for monitoring air flow through the first and second troughs, wherein the visual/audio indicator comprises a micro total analysis system for detecting, recording, and reporting detected pathogens.

16. A decontaminating scrubber filter table as in claim 15 wherein the scrubber further comprises a negative ion generator.
